(19) United States
(12) Patent Application Publication
(10) Pub. No.: US 2017/0273574 A1
(43) Pub. Date: Sep. 28, 2017

(54) WEARABLE PHYSIOLOGICAL MEASURING DEVICE

(71) Applicant: Hong Yue Technology Corporation, Hsinchu City (TW)

(72) Inventors: Chi-Sheng Wu, Hsinchu City (TW); Yu-Sheng Liao, Hsinchu City (TW); Mei-Hua Liao, Hsinchu City (TW)

(73) Assignee: Hong Yue Technology Corporation, Hsinchu City (TW)

(21) Appl. No.: 15/469,599

(22) Filed: Mar. 27, 2017

(30) Foreign Application Priority Data
Mar. 25, 2016 (TW) 105109595

(51) Int. Cl. A61B 5/0205; A61B 5/021; A61B 5/0408; A61B 5/145; A61B 5/0456; A61B 5/00

(52) U.S. Cl. CPC A61B 5/02055; A61B 5/0456; A61B 5/6823; A61B 5/4875; A61B 5/6824; A61B 5/6829; A61B 5/04085; A61B 5/0002; A61B 5/486; A61B 5/14542; A61B 5/746; A61B 5/021; A61B 2562/0219; A61B 2560/0242; A61B 2560/0223; A61B 5/02416

(57) ABSTRACT

A wearable physiological measuring device has a torso-worn module and a limb-worn module configured to communicate in a wireless way with each other. The torso-worn module is configured to be coupled with a torso of a user to obtain an R-peak from an electrocardiac signal. The limb-worn module is configured to be coupled with at least one limb of four limbs of the user to obtain a pulse wave peak from a plethysmograph signal. There are no physical connections (neither wire nor cable) between the torso-worn module and the limb-worn module. The wearable physiological measuring device is configured to use the R-peak time and the pulse wave peak time to generate a pulse transit time data.

### BACKGROUND OF THE INVENTION

Field of the Invention

The present invention relates to a wearable physiological measuring device and a physiological measuring method.

Description of Related Art

The principle of continuous non-invasive blood pressure, CNIBP, is specified in a paper “A review of methods for non-invasive and continuous blood pressure monitoring: Pulse transit time method is promising?” by Peter et al published in IRBM, 2014. Nowadays, a frequently-used method is to measure the pulse transit time, PTT, which is the time that a pulse starts to propagate from left ventricle (marked by the R-peak of electrocardiogram, ECG, which propagates at speed of light) to any one of the four limbs (marked by the peak observed by tonometry, electro-impedance plethysmograph (IPG), or photo plethysmograph (PPG)). By PPT and supplemented by other hemodynamics parameters such as elasticity of blood vessels and viscosity of blood, refer to U.S. Pat. No. 5,865,755 A, the CNIBP can be calculated. The relation of viscosity of blood and blood pressure can be specified by Hagen-Poiseuille equation ΔP=8uLQ/πr⁴, where ΔP is the pressure reduction, L is the length of blood vessel, u is the blood viscosity, Q is the volumetric flow rate, r is the vessel radius. Hagen-Poiseuille equation shows that the blood pressure is proportional to blood viscosity. However, the aforementioned hemodynamics parameters, such as elasticity of blood vessels and viscosity of blood, are not easy to measured directly; therefore the blood pressure by traditional cuff-type non-invasive blood pressure meter, NIBP, needs to be measured simultaneously together with PTT before long-term CNIBP can be measured, to obtain the aforementioned hemodynamics parameters as calibration standards. After the calibration standards are set, PTT can be used to calculate CNIBP for a period of time, e.g., two hours. After the period, the hemodynamics parameters may varied significantly by sweating, urinating, environmental temperature and humidity, etc. such that NIBP needs to be measured again to revise the calibration standards, hence the accuracy of CNIBP can be maintained. For example, water content will decrease significantly after sweating a great amount such that the blood viscosity increases significantly, and hence the blood pressure.

To enable users to measure CNIBP during daily life routines without being constrained by a blood pressure meter, the CNIBP meter is preferred to be worn on body or limbs. For example, a series of patents such as U.S. Pat. No. 8,475,370 B2 and U.S. Pat. No. 8,364,250 B2 granted to Sotera Wireless disclose techniques wherein three electrodes are pasted on a user's chest to acquire R peaks from ECG signals (which marks the time that a pulse starts to propagate from left ventricle) and connected with cables which pass over left shoulder and bonded to a main machine fastened on left arm; a PPG sensor comprising a LED and a photo sensor is worn on left thumb to acquire peaks from PPG signals (which marks the time that a pulse propagate to left thumb) and connected with cables bonded to the said main machine. From the time interval of the aforementioned peaks of ECG and PPG, i.e., PTT, can be calculated. In addition, a traditional cuff type NIBP meter is also temporary worn on user's upper arm for calibration and is detached after calibration thus that user can feel comfortable and is not ridden. The said patents are realized to be a commercially available product named ViSi Mobile Monitoring System and cleared by USFDA with clearance number K130709. However, the aforementioned techniques are involved with pasting electrodes on specific site on human body, which are not comfortable at all and quite difficult for ordinary personnel, hence it should be operate by clinician. Furthermore, USFDA permits it can only be used in medical site and only with physician's prescription. U.S. Pat. No. 7,993,275 B2 also granted to Sotera Wireless discloses a handheld apparatus that acquires two PPG signals and ECG to calculate PTT from both hands to increase accuracy.

U.S. Pat. No. 7,896,811 B2 granted to Samsung discloses a handheld apparatus wherein electrodes and tonometry plethysmograph sensor are installed on a mobile phone to acquire ECG and pulse signals. However, the user can do nothing by hands while holding this apparatus.

In summary, current technologies provide an apparatus that is connected to ECG electrodes and plethysmograph sensor. To measure CNIBP with this apparatus, long electric cables are necessary such that it makes the user feels uncomfortable and not willing to accept it. The current technologies fail to provide a comfortable and easy-to-operate wearable PTT measurement device to the confirmed and potential patients with hypertension and stroke for long term use, to acquire real time and continuous physiological information.

### SUMMARY OF THE INVENTION

The objective of the present invention is to provide a wearable physiological measuring device, which comprises a torso-worn module and a limb-worn module. There are no physical connections (neither wire nor cable) between the torso-worn module and the limb-worn module. The torso-worn module and the limb-worn module are configured to communicate with each other in a wireless way. The torso-worn module is configured to be coupled with a torso of a user to obtain an R-peak from an electrocardiac signal. The limb-worn module is configured to be coupled with at least one of four limbs of the user to obtain a pulse wave peak from a plethysmograph signal. The present wearable physiological measuring device is configured to use the R-peak and the pulse wave peak to generate a pulse transit time data.

In one embodiment of the present invention, the torso-worn module or the limb-worn module of the wearable physiological measuring device is configured to wirelessly transmit the pulse transition time to an information/telecommunication technological (ICT) equipment.

Another objective of the present invention is to provide a method for obtaining a pulse transit time data. The method comprises a providing step, an R peak time acquiring step, a pulse wave peak time acquiring step, and a pulse transit time data generating step. The providing step provides a wearable physiological measuring device, which comprises a torso-worn module and a limb-worn module. The torso-worn module is configured to be coupled with a torso of a user, and the limb-worn module is configured to be coupled with at least one of four limbs of the user. There are no physical connections (neither wire nor cable) between the torso-worn module and the limb-worn module. The torso-worn module and the limb-worn module are configured to wirelessly communicate with each other. The R-peak time acquiring step uses the torso-worn module to obtain a R-peak time from an electrocardiac signal, and the pulse peak time acquiring step uses the limb-worn module to obtain a pulse wave peak time from a plethysmograph signal. In the pulse transit time data generating procedure, the present wearable physiological measuring device generates a pulse transit time data according to the R-peak time and the pulse wave peak time.

### BRIEF DESCRIPTION OF THE DRAWINGS

FIG. 1 schematically shows a wearable physiological measuring device and an example of its wireless communication according to one embodiment of the present invention.

FIG. 2 shows the detailed structure of a torso-worn circuit of a wearable physiological measuring device according to one embodiment of the present invention.

FIG. 3-1 shows the detailed structure of a limb-worn circuit of a wearable physiological measuring device according to one embodiment of the present invention.

FIG. 3-2 shows the detailed structure of a limb-worn circuit of a wearable physiological measuring device according to another embodiment of the present invention.

FIG. 3-3 shows the detailed structure of a limb-worn circuit of a wearable physiological measuring device according to yet another embodiment of the present invention.

FIG. 4 shows various aspects of a torso-worn module according to one embodiment of the present invention.

FIG. 5 shows a schematic top view of a torso-worn module.

FIG. 6 shows a schematic rear side view of a torso-worn module.

FIG. 7-1 shows a side view of a status before electrodes are coupled with the torso belt according to one embodiment of the present invention.

FIG. 7-2 shows a side view of a status after electrodes are coupled with the torso belt according to one embodiment of the present invention.

FIG. 8 shows a side view of a status when electrodes and the torso belt are applied to a human body according to another embodiment of the present invention.

### DESCRIPTION OF THE INVENTION

The wearable physiological measuring device of the present invention uses an easy-to-wear and comfortable torso-worn module in which ECG electrodes installed and a limb-worn module in which a plethysmograph sensor installed, wherein there are no physical connections (neither wire nor cable) between the torso-worn module and the limb-worn module. Instead, both modules communicate wirelessly to obtain pulse transit time data (PTT data) to calculate continuous non-invasive blood pressure (CNIBP). Hence, the present invention can monitor the physiological conditions of a user invasively and continuously without interfering sleep or daily routine at all, comparing with the current techniques of which the device used for monitoring is neither comfortable nor easy to wear on. Besides, the present invention additionally includes other sensor to exclude body motion interference, and to remind the user to calibrate once again whenever the hemodynamics parameters vary significantly in order to maintain the accuracy of measurement.

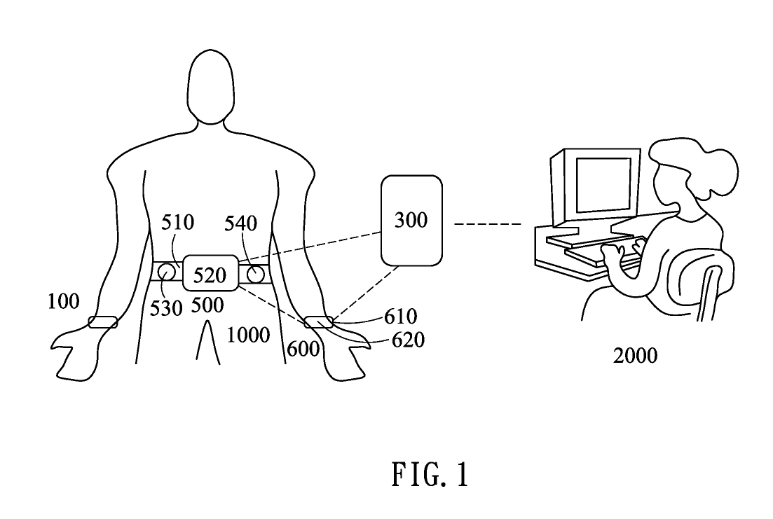

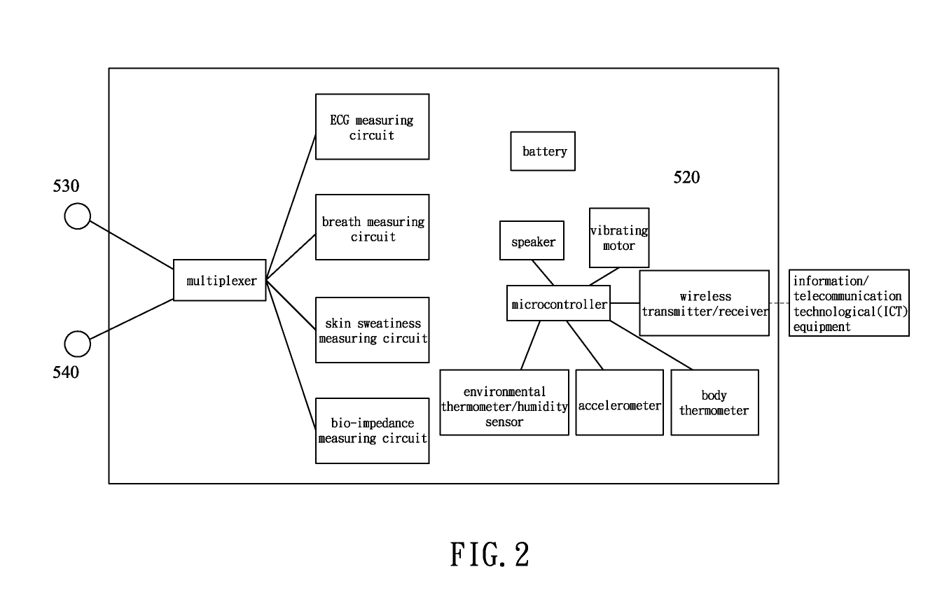

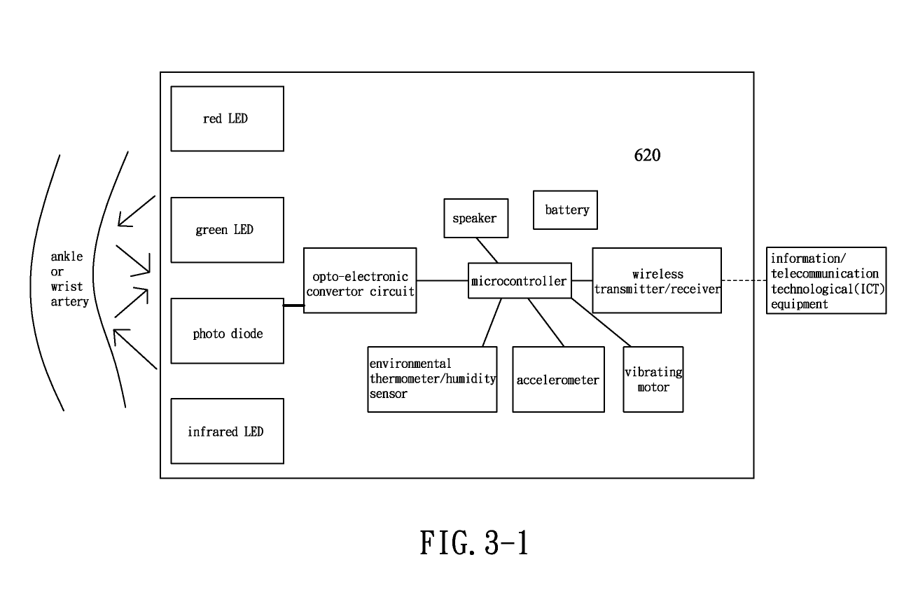

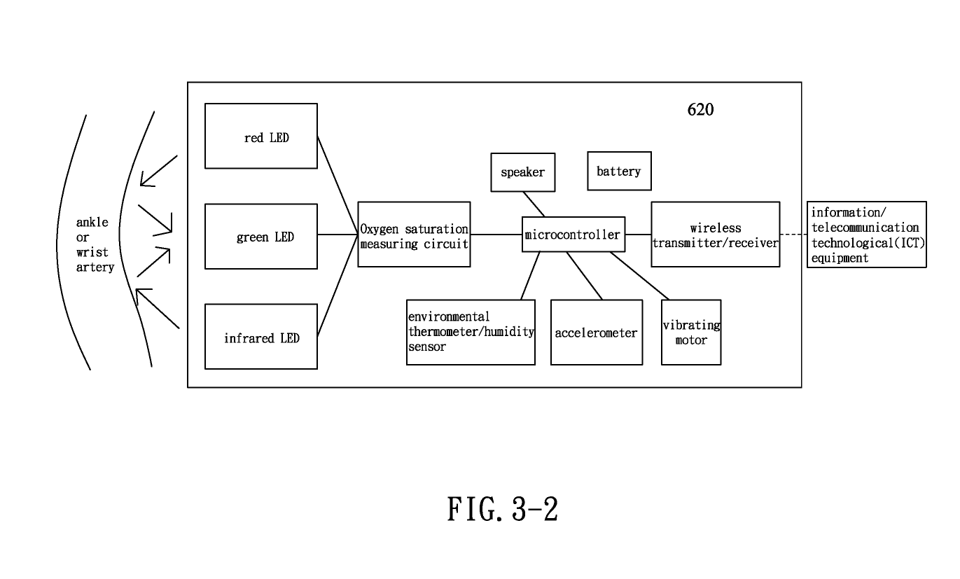

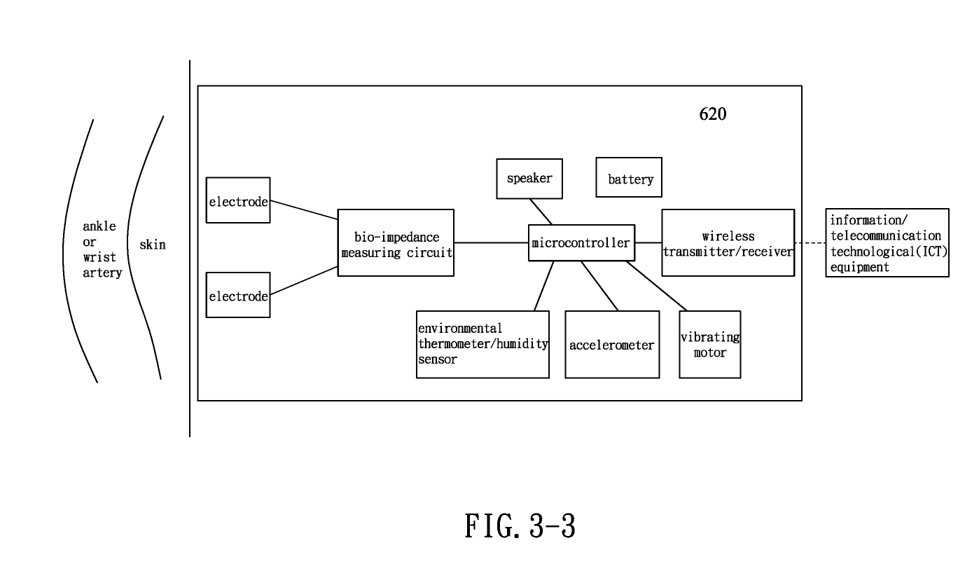

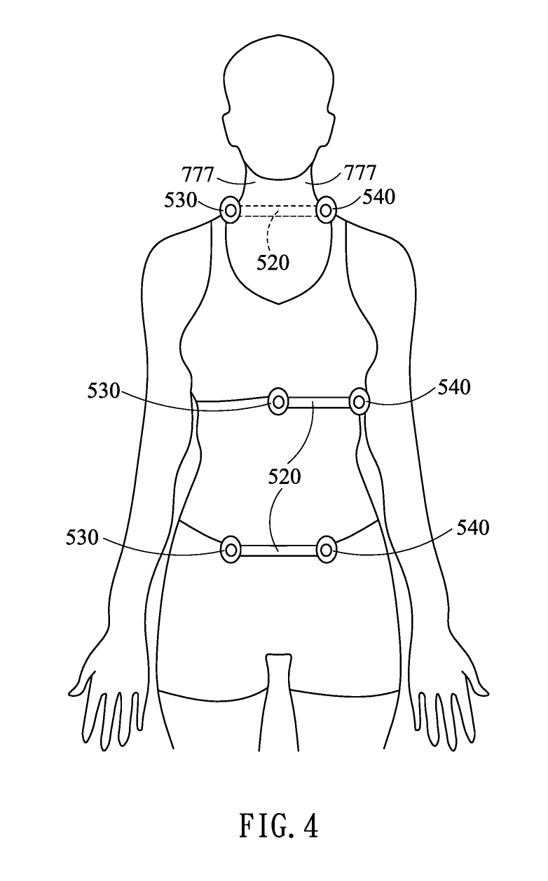

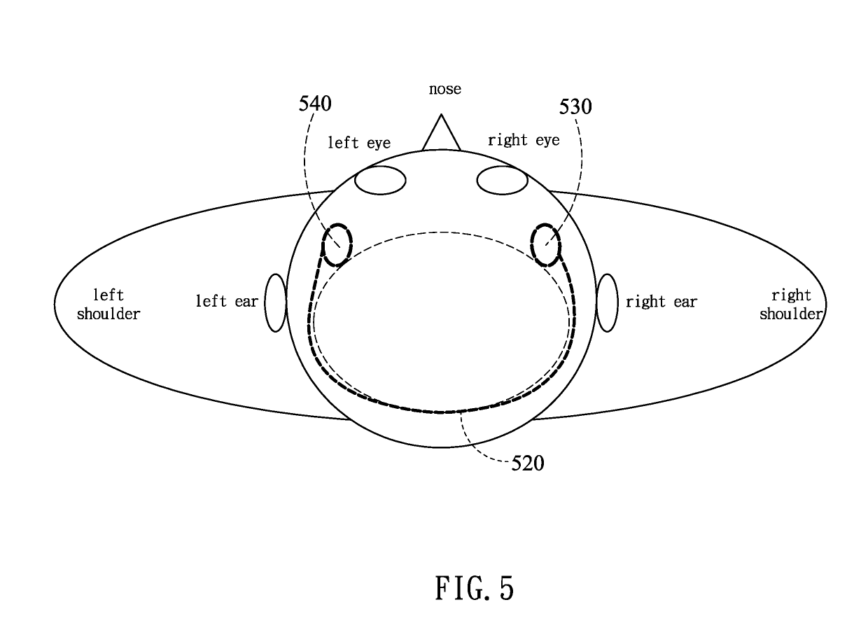

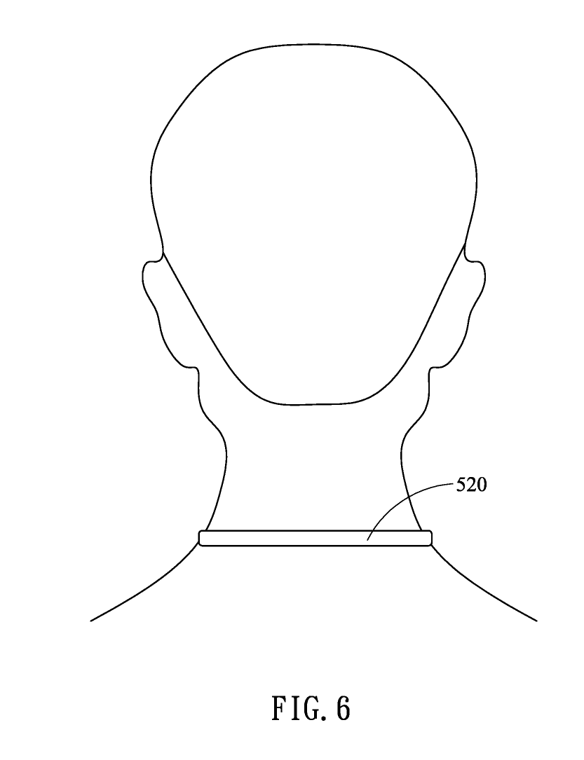

Please refer FIG. 1 to FIG. 6. FIG. 1 schematically shows the wearable physiological measuring device 1000 and an example of its wireless communication according to one embodiment of the present invention. FIG. 2 shows the torso-worn circuit 520 of the aforementioned 1000, according to one embodiment of the present invention. FIG. 3-1, 3-2, 3-3 show detailed structures of limb-worn circuit 620 of the aforementioned 1000 according to one embodiment of the present invention. FIG. 4 shows various aspects of the torso-worn module according to one embodiment of the present invention. FIG. 5 shows a schematic top view of the torso-worn module. FIG. 6 shows a schematic rear side view of the torso-worn module. As shown in FIG. 1, the wearable physiological measuring device 1000 comprises a torso-worn module 500 and a limb-worn module 600. The torso-worn module 500 comprises a torso belt 510 and a torso-worn circuit 520, wherein the torso belt 510 may be a rubber band waist belt, a leather belt, or any kind of holder that can be fastened on a torso, such as a brief, a brassiere, of a necklace. Two fixed or detachable electrodes, 530 and 540, are installed on the torso belt 510 to contact the skins of a wearer's abdomen, chest, or neck at the left side and at the right side of a heart, as shown in FIGS. 1 & 4, FIG. 4, and FIGS. 5 & 6, respectively. FIG. 4 shows the front side of a wearer's whole body, from which the electrodes 530 and 540 worn on neck can be seen, while the other electrode 520 worn on rear side cannot been seen, hence it is presented by dash line. FIG. 5 shows a top view of a wearer's head and shoulder with the torso-worn module worn on neck; because the neck is sheltered by the head and hence cannot be seen, it is presented by dash line. In FIG. 5, a C-shape torso belt 510 made of plastic with a little elasticity to be fastened on neck is presented by a fine dash line, and the torso circuit 520 fixed on the torso belt 510 is presented by course dash line, where electrodes 530 and 540 are installed at both ends of torso belt 510. FIG. 6 shows the rear view of a wearer's neck, where the torso belt 510 and torso circuit 520 are attached to and both electrode 530 and 540 installed at both ends of torso belt 510 are not seen.

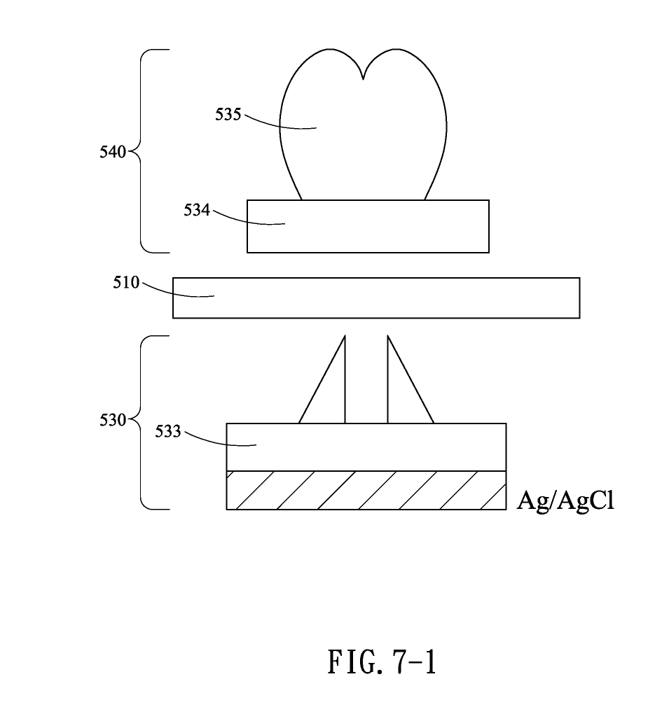

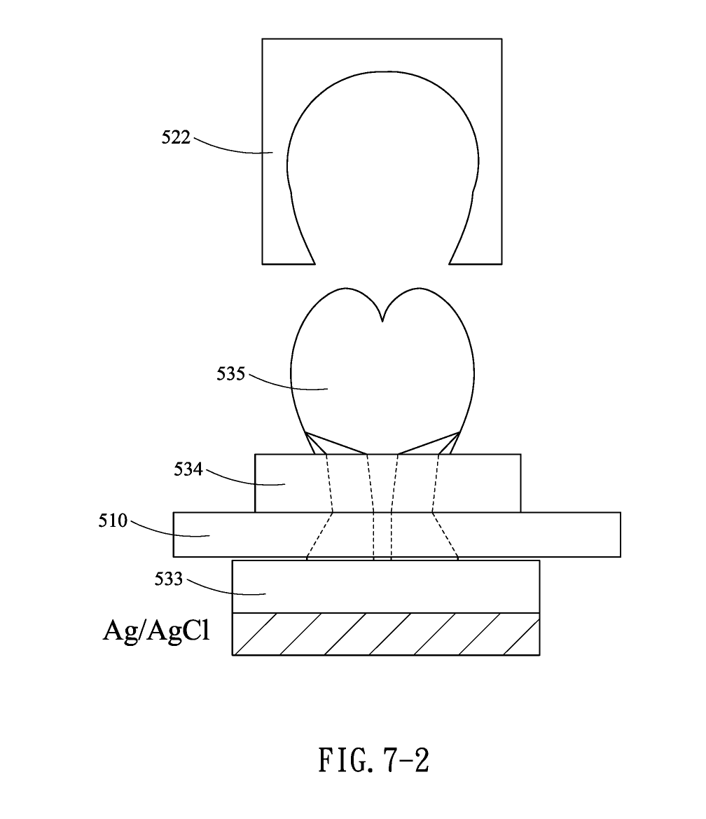

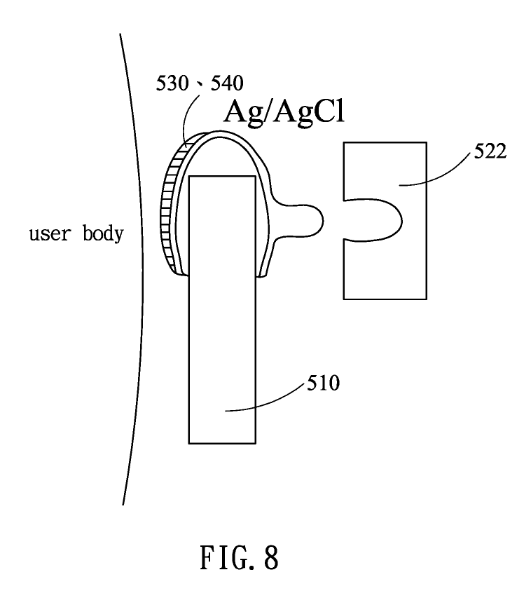

The electrodes 530 and 540 can be coupled with torso belt 510 by various ways. Please refer FIGS. 7-1, 7-2, and 8. FIGS. 7-1 and 7-2 show side views of statuses before and after electrodes 530, 540 are coupled with torso belt 510 according to one embodiment of the present invention. FIG. 8 shows a side view of a status when electrodes 530, 540 and torso belt 510 are applied to a human body according to another embodiment of the present invention. In the situation that the electrodes 530 and 540 are fixed on the torso belt 510, the main bodies of the electrodes 530 and 540 may be prong snap buttons which are widely used in textile industry, which are comprised of bottom metal plate 533 and top metal plate 534, of which the side view is shown in FIG. 7-1 before they are fastened. The bottom metal plate 533 attached onto wearer's body has sharp claws to punch through torso belt 510 so as to engage with the top metal plate 534. The top metal plate 534 comprises a protruding cavity 535 that acts as a male connector of a push button to contain the sharp claws of bottom metal plate 533. While installing metal plates 530 and 540 on torso belt 510, both metal plates are pushed to each other thus that the protruding cavity 535 squeezes the sharp claws aside such that both metal plates are engaged closely and clip on torso belt 510, the side view of which is shown in FIG. 7-2. As shown in FIG. 8, the protruding cavity 535 is engaged and electrically connected with female connector 522, and the surfaces of electrode 540 and 530 that contact human body are preferably coated with a silver layer and a silver chloride layer (Ag/AgCl) to maintain a stable chemical electrical potential, thus high quality ECG signal can be obtained.

In the situation that electrode 530 and 540 are detachable, both electrodes can be male push buttons made of conductive material where they look like a U-shape by side view, as shown in FIG. 8. Push buttons are clamped on both sides of torso belt, e.g., brief or brassiere, where the plane surfaces of the push buttons are used to contact with human body and are preferably coated with a silver layer and a silver chloride layer (Ag/AgCl), as mentioned. The male sides of the push buttons are used to engage with the female connector 522 extended from torso circuit 520. More specifically, the male side of electrode 530 is used to engage with the female connector 522 extended from torso circuit 520, and he male side of electrode 540 (not shown) is used to engage with the female connector (not shown) extended from torso circuit 520. The male sides of electrode 530 and 540 are compatible with the female connector of cable extended from physiological monitors that are popular in hospitals, such that electrode 530 and 540 can be connected to hospital physiological monitors when necessary. Torso belt 510 tied at waist or chest may be made of extendable fabrics, such as Lycra which contains Spandex thus that it can be washed by laundry machine and is also comfortable and to locate electrodes 530, 540 and torso circuit 520 on the wearer's torso. Torso belt 510 may also be a modified leather belt so that it is convenient for the wearer to put on when working or going outside. Regarding to the electrodes 530 and 540 situated at abdomen, the ECG amplitude obtained would be too small to identify R-peaks if the electrodes 530 and 540 are too close to each other. In the present invention, a mid-size (about 160 cm height) adult is tested as subject. The ECG amplitude obtained by two electrodes with a distance more than 10 cm is 0.3 mV, which is good enough to identify R-peaks. 10 cm distance is also suitable for electrodes 530 and 540 situated at middle and left side of brassier, as shown in FIG. 4, hence that the same torso circuit 520 can be suitable for both abdomen and chest.

The torso belt 510 positioned on the neck may be a plastic-made C-shape clip as shown in FIG. 5 and FIG. 6, wherein electrodes 530 and 540 are installed at both ends of the torso belt 510 with torso belt 510 applying a tiny force on both sides of wearer's neck. There are two purposes to realize torso belt 510 as a C-shape clip: first, others can see only a small part of C-shape clip from front side view, thus the wearer does not look ugly; second, C-shape clip can be easily pulled away from the wearer's neck, thus the wearer would not be choked even if the C-shape clip were hooked by other subject unintentionally. The torso belt 510 positioned on the neck is preferably situated close to the heart, thus the amplitude of the obtained ECG signal can be larger, and the distance to the right and left carotid sinus can also be larger, as shown in FIG. 4. Carotid sinus is the blood pressure sensors of the blood pressure regulation mechanism of human body, once it is pressed the blood pressure might be unstable or even so low to cause low pressure shock. To locate the torso belt 510 far away from the right and left carotid sinus will give less effect on blood pressure and be more comfortable to the wearer.

Please refer to FIGS. 1 to 6. As shown in FIG. 2, torso circuit 520 comprises an ECG measuring circuit, a wireless transmitter/receiver, a microcontroller, and female push button (not shown), to acquire ECG signal to detect R peaks, and then the time of R peaks are transmitted through the wireless transmitter/receiver to limb circuit 620 (shown in FIG. 3) or other information/telecommunication technological (ICT) equipment 300. Torso circuit 520 can optionally include breath measuring circuit, skin sweatiness (i.e., Galvanic Skin Response, GSR) measuring circuit, bio-impedance measuring circuit to measure water content, and multiplexer (coupling with electrode 530 and 540), in addition. Limb module 600 comprises limb circuit 620, and limb belt 610 such as wrist ring, watch belt, ankle ring, sock, or any kind of fastener that can situate on wrist or ankle to attach limb circuit 620 on the wearer's wrist or ankle. Wrist and ankle are better sites for PPG sensor than others because the skins at wrist and ankle are thinner and have almost no fat and muscle located between artery and PPG sensor, therefore stronger signal and less interference are obtained by PPG sensor. As shown in FIG. 3-1, limb circuit 620 comprises a source of light that can be reflected or absorbed by red blood cell, such as a green, red, or infrared LED, a photo sensor that receives the light reflected or not absorbed by red blood cell, such as a photo diode, an opto-electronic convertor circuit coupling to the photo-sensor to convert the light into electronic signal, a wireless transmitter/receiver circuit, a microcontroller, and a battery. Limb circuit 620 is configured to acquire pulse wave signal from plethysmogram and define the time of pulse and then subtract it with the time of the R peak from ECG obtained by torso circuit 520 to obtain the PTT, which can be transmitted by the wireless transmitter/receiver circuit to the ICT device 300, such as cellular phone, health monitor, signal relay, etc., preferable a cellular phone. Alternatively, torso circuit 520 does calculation to obtain the PTT and transmit the PTT to the ICT device 300. Torso circuit 520 and limb circuit 620 are preferably based on flexible printed circuit, on which the said electronic components are soldered and polymer materials with good biocompatibility such as silicone or polyurethane are coated to protect the said electronic components and give elasticity enough to let the wearer feel comfortable.

Please refer FIG. 1. When beginning to monitoring CNIBP, ICT device 300, such as cellular phone, may prompt the user to use a conventional blood pressure meter 100 and the wearable physiological measuring device 1000 proposed by the present invention simultaneously to calibrate and to acquire and save the hemodynamic parameters necessary for calculating CNIBP. After calibrating, ICT device 300 can receive the PTT transmitted from torso circuit 520 or limb circuit 620 to calculate and display CNIBP. The user can set the normal range of CNIBP in advance, thus that whenever the measured CNIBP ran out of the preset normal range, ICT device 300 may launch alarm such as special ringing, vibrating with ringing, flashlight with ringing or text information to the user or nearby care giver, or send a text message, an email or a phone call to the remote monitoring center 2000 to make sure that at least one of the parties mentioned above would take necessary action. Torso module 500 and limb module 600 are separate and no physical wire connected in between to interfere user action and then they are comfortable enough to be worn while sleeping and will not affect daily routines. Thus, the wearable physiological measuring device 1000 proposed by the present invention can continuously monitor blood pressure of a wearer in daily routine activities and measure other physiological parameter simultaneously. The preferred embodiments specified in detail and their applications are recited below.

### First Embodiment: Applying Wearable Physiological Measuring Device to CNIBP

As shown in FIG. 2, a multiplexer such as 74HC4052 can be additionally installed in torso circuit 520 to select one of the three optionally installed circuits: ECG and respiration measuring circuit (e.g., Texas Instruments ADS1292R), Galvanic skin response (GSR) measuring circuit (to measure the resistance of skin by DC bias), and bio-impedance circuit (to measure water content by a tiny high frequency AC current, e.g., Texas Instruments AFE4300). Besides, an environmental thermometer/humidity sensor (e.g., Texas Instruments HDC1008), a body thermometer closely contacted to human skin (e.g., Texas Instruments LMT70), and an accelerometer (e.g., Freescale MMA8652) can also be installed in torso circuit 520 to detect skin temperature and human body activity. On the other hand, alarm devices such as vibration motor and/or speaker to generate sensory effect warning signals like vibration, beep, and/or voice to alert the user or nearby care giver can also be included in torso circuit 520. For example, when the accelerometer sense the human body stays still without action, torso circuit 520 begins to acquire ECG signal and detect R peaks by a well-known algorithm, e.g., So and Chan, and wirelessly transmit the times of R peaks to limb circuit 620 through Bluetooth or similar techniques. On the same time torso circuit 620 detects pulse peaks from plethysmograph to define the times of pulses arriving to calculate PPT, and then calculate CNIBP by the aforementioned algorithm. Or, limb circuit 620 transmits the times of pulses arriving to torso circuit 520, so that torso circuit 520 can calculate CNIBP.

Beside, torso circuit 520 can undergo heart rate variability analysis (HRV, refer to Camm et al: “Heart Rate Variability: Standards of Measurement, Physiological Interpretation, and Clinical use.” Circulation, 93, 1043-1065, 1996) by the times of R peaks. After continuously measuring ECG for three to five minutes, torso circuit 520 can measure sweatiness (i.e. GSR) and bio-impedance to measure water content. When the wearer is in light action, e.g., speaking or eating, torso circuit 520 can still transmit the times of R peaks to limb circuit 620, while it is not necessary to undergo HRV analysis (because HRV is valid only when body is not in action). When the wearer is in heavily action such as running or climbing upwards on stairs, torso circuit 520 may stop capturing ECG because ECG signal would be so badly interfered to identify R peaks; however, environmental temperature and humidity can still be measured. By measuring skin sweatiness and bio-impedance, whether or not the water content of the wearer varied drastically can be determined. As water content varies, the viscosity of blood varies accordingly, and then the hemodynamic parameters vary. By measuring environmental temperature and humidity, skin temperature and sweatiness, the effect on peripheral blood vessels by environment can be evaluated. By HRV analysis, whether or not the wearer is under major mental stress can be determined. For example, the heart rate data can be Fast Fourier Transformed (FFT) to obtain its Low Frequency (LF, 004˜0.15 Hz) and High Frequency (HF, 0.15˜0.4 Hz) power spectrum density. When the LF/HF ratio decreasing, it means that the mental stress and the activity of sympathetic nerve of the wearer are decreased, the blood pressure is also decreased accordingly. Whenever these physiological and environmental parameters changed significantly to be out of the pre-set or default normal range after calibration, it means that the hemodynamic parameter that set by former calibration are no long suitable, and then the torso circuit 520 turns on the alarm devices such as vibrating motor or speaker or other alerting mechanisms above mentioned to generate sensory effect to remind the user to calibrate once again by conventional NIBP. Besides, the user may change the environmental temperature by turn on an air conditioner or a heater, or change the undergoing routine activity such as giving a brief stop on a busy and tight working schedule so to avoid hypertension which might prejudice the user's health. Torso circuit 520 may send the message that physiological or environmental parameters run out of normal range via wireless transmission to limb circuit 620 or nearby ICT device 300 such as cellular phone or panel computer to generate a vibrating, sound, or video signals to remind the user.

On the other hand, by calculating PPG signal by receiving the reflected light from any one of green, red, or infrared LED, Vessel Dilation Index (VDI, Taiwan Patent 1473595) or Augmentation index (AI, U.S. Pat. No. 6,786,872 B2) can be obtained to evaluate the condition of vessels and blood pressure. Similar to the processes for environmental temperature, humidity, skin temperature, GSR, and HRV by torso circuit 520 shown above, limb circuit 620 can also judge whether any one of the VDI, AI, environmental temperature and humidity has changed so significantly after calibration that they go out of its default normal range or the range pre-defined by the user. Whenever they go out of range, limb circuit 620 will turns on the alarm devices such as vibrating motor or speaker or other alerting mechanisms above mentioned to generate sensory effect to remind the user or a near-by care-giver to calibrate once again by conventional NIBP. For another option, limb circuit 620 may send the message that physiological or environmental parameters run out of normal range to nearby ICT device 300 such as cellular phone or panel computer to generate a vibrating, sound, or video signals to remind the user or near-by care-giver.

By co-operating torso circuit 520 and limb circuit 620, PTT, environmental temperature, humidity, skin temperature, sweatiness (GSR), water content, and HRV can be measured and transmitted to a nearby ICT device 300 such as cellular phone or panel computer. The ICT device 300 can not only calculate CNIBP, but also can judge whether the measured data is normal or not by the default or pre-defined normal range, and then launch warning alarm such as vibration, sound, or video to remind the user or nearby care-giver to do necessary intervention such as turning on air conditioner heater, or using a traditional NIBP machine to recalibrate the system.

The ICT devices can also transmit the measured data to internet access point, then a remote monitoring center for storage and further analysis. When necessary, the clinicians in the remote monitoring center can instruct the user or nearby care-giver to give necessary treatment such as taking hypertension medicine or visiting hospital, etc.

### Second Embodiment: Applying Wearable Physiological Measuring Device to Monitor CNIBP and Co-Operate with Guided Respiration to Regulate Blood Pressure

It is well known that the major resistance of blood flow is given by peripheral blood vessel, specifically, arteriole, of which dilation or contraction is regulated sympathetic nerve. Because of the antagonism of sympathetic and parasympathetic nerve, parasympathetic nerve can be strengthened by deep breathing to stimulate the receptor of parasympathetic nerve in diaphragm, therefore deep breathing can lower blood pressure. This method is known as biofeedback blood pressure regulation, which is disclosed in U.S. Pat. No. 5,800,337 and commercialized as Resperate® that cleared by USFDA with pre-market notification number K020399. The configuration of the wearable physiological measuring device is similar to the one specified in preferred embodiment 1, but additionally its torso circuit 520 wirelessly transmits the pneumograph signal acquired by its ECG and respiration measurement circuit to nearby ICT device such as cellular phone or panel computer, in which a program can be installed to judge whether the received CNIBP is larger than the upper limit of the pre-determined normal range. If larger, the cellular phone or panel computer may guide the user by audio or visual message such as voice instruction, text and voice instruction, or visual and voice instruction to apply biofeedback to lower blood pressure to prevent hyper tension from jeopardizing health.

### Third Embodiment: Applying Wearable Physiological Measuring Device to Monitor Sleep Apnea and Rapid Eye Movement (REM) Period

The present invention can also be applied to monitor sleep apnea as well. During sleep, torso circuit 520 and limb circuit 620 can not only calculate CNIBP and HRV and wirelessly transmit the data to a nearby ICT device such as cellular phone or panel computer, but also transmit other physiological parameters such as acceleration, respiration, Oxygen saturation (SpO2), and sweatiness (GSR). The ICT device can also record video image and snoring sound simultaneously thus that it can record physiological variations of the whole sleep period. When sleep apnea happens, breath stops temporary, SpO2 decreases, and other physiological parameters may be abnormal. All these parameters and signals can be recorded by the present invention as reference information for screening test of sleep apnea and further diagnosis and therapy by physicians. Besides, when limb circuit 620 detects that the SpO2 is lower than normal, e.g., 90%, or torso circuit 520 detects that sleep apnea continues a longer time than normal, e.g., 15 seconds, a warning signal device such as a vibration motor can be turn on to wake up the user to avoid hypoxia for a long time.

The present invention can also be applied to observe the Rapid Eye Movement phase (REM) during sleep. Usually a normal human adult sleeps about eight hours per day, which can be divided into four similar sleep cycles, two hours in average for each cycle. In one sleep cycle the sleeper experienced gradually from REM phase, stage one phase (least-deep), stage two, stage three, then stage four (deepest sleep); and then stage three, two, one, finally back to REM phase, and then a new sleep cycle begins. In the first phase of a sleep cycle, sleeper frequently move slightly such as roll-over or limbs movement, and his/her eyes also move rapidly simultaneously, thus it is known as REM. To detect REM, eye movement can be observed directly, or an accelerometer can be used to observe limbs movements. Besides, HRV can also be applied to detect REM, referring “Power spectrum analysis and heart rate variability in Stage 4 and REM sleep: evidence for state specific changes in autonomic dominance._{┘} J. Sleep Res 1993; 2 (2)” by Berlad et al. The low frequency power spectrum density (LF) of REM is significantly higher, while high frequency power spectrum density (HF) of non-REM is significantly higher. It is well known that a sleeper would feel less tired if awakened during REM, while would feel very tired and not happy if awakened during non-REM. The present invention can determine whether the sleeper is in REM or not by the accelerometer installed in torso circuit 520 or limb circuit 620 to detect body movement, or by torso circuit 520 to analyze HRV. The user can set the time interval suitable for awakening, e.g., AM 6:30˜7:30, then the present invention will determine the REM of the sleeper and be alarming by the vibration motor or the speaker installed in torso circuit 520 or limb circuit 620 to awake the sleeper.

Those who are familiar with the art should understand that the present invention is not limited to the specific components described in the above embodiments; the present invention can use any other components or devices that can give the same functions to replace the components described above. For example, the short range wireless communication between torso module 500 and limb module 600 can use Wi-Fi, ZigBee, UWB or other techniques; the long range wireless communication between wearable physiological measuring device 1000 and remote monitoring center 200 can use GSM, 3G, 4G/LTE or other techniques. Besides, the names of components do not mean the shapes and/or sizes. For example, “circuit box” is not limited to a cube; it can also be a flat cylinder, an ellipsoid, or a flat panel similar to a card or an IC chip. In addition, the IC chips listed above, such as the ones made by Texas Instruments or Freescale, can be replaced by the IC chips with similar functions but made by other manufacturers. The wearable physiological measurement device specific in the present invention is not limited to be applied in the above embodiments; it can be applied to all the applications related to ECG signals and/or plethysmograph pulse signal that are already known in the past or would be developed in the future.

Although the invention has been described with reference to specific embodiments, this description is not meant to be construed in a limiting sense. Various odifications of the disclosed embodiments as well as alternative embodiments of the invention will become apparent to persons skilled in the art. It is therefore contemplated that the appended claims will cover any such modifications or embodiments that fall within the scope of the invention.

What is claimed is:

1. A wearable physiological measuring device, comprising:
a torso-worn module configured to be coupled with a torso of a user to obtain a time of an R-peak from an electrocardiac signal, wherein the torso-worn module comprises a torso belt and a torso circuit which includes a water content measuring circuit to measure a water content of the user; and
a limb-worn module configured to be coupled with a wrist or an ankle of the user to obtain a time of a pulse wave peak from a plethysmograph signal,
wherein the torso-worn module and the limb-worn module are configured to communicate with each other in a wireless way without physical connections or cables therebetween,
wherein the wearable physiological measuring device is configured to create a pulse transit time data from the time of the R-peak from the electrocardiac signal and the time of the pulse wave peak from the plethysmograph signal.

2. The wearable physiological measuring device according to claim 1, wherein the torso belt comprises two electrodes configured to contact respectively skins at right side and at left side of a hear of the user.

3. The wearable physiological measuring device according to claim 1, wherein the torso circuit further comprises an electrocardiac amplifier, a microcontroller, a wireless transmitter/receiver circuit, and a battery.

4. The wearable physiological measuring device according to claim 1, wherein the limb-worn module comprises a limb belt and a limb circuit.

5. The wearable physiological measuring device according to claim 4, wherein the limb circuit comprises a light source, a photo sensor, an opto-electronic convertor circuit, a microcontroller, a wireless transmitter/receiver, and a battery.

6. The wearable physiological measuring device according to claim 1, wherein said torso-worn module is configured to wirelessly transmit the pulse transit time data to an information/telecommunication technological equipment.

7. The wearable physiological measuring device according to claim 1, wherein said limb-worn module is configured to wirelessly transmit the pulse transit time data to an information/telecommunication technological equipment.

8. The wearable physiological measuring device according to claim 6, wherein said information/telecommunication technological equipment is configured to wirelessly transmit the pulse transit time data to a remote monitoring center for storing and analyzing so as to direct the user or a care-giver of the user, wherein said information/telecommunication technological equipment is a cellular phone.

9. The wearable physiological measuring device according to claim 8, wherein said information/telecommunication technological equipment is configured to send a sensory effect to guide the user to regulate respiration rhythm, wherein said information/telecommunication technological equipment is a cellular phone.

10. The wearable physiological measuring device according to claim 1, wherein said limb circuit further comprises one or more of another light source, a blood oxygen saturation circuit, an accelerometer, a thermometer, an ambient humidity sensor, and a warning message generator.

11. The wearable physiological measuring device according to claim 10, wherein said warning message generator is configured to be turned on while a measured water content of the user goes beyond a pre-determined range.

12. The wearable physiological measuring device according to claim 11, wherein said wearable physiological measurement device is configured to remind the user by a sensory effect to use a non-invasive blood pressure meter for calibrating while the measured water content goes beyond the pre-determined range.

13. A method for obtaining a pulse transit time data, comprising:
providing a wearable physiological measurement device which comprises a torso-worn module configured to couple to a torso of a user and a limb-worn module configured to couple to a wrist or an ankle of the user, wherein the torso-worn module comprises a torso belt and a torso circuit which includes a water content measurement circuit to measure a water content of the user, wherein the torso-worn module and the limb-worn module are configured to communicate with each other in a wireless way without physical connections or cables therebetween;
using the torso-worn module to acquire a R-peak time from an electrocardiac signal;
using the limb-worn module to acquire a pulse peak time from a plethysmograph signal; and
generating a pulse transit time data according to said R-peak time and said pulse peak time.

14. The method for obtaining a pulse transit time data according to claim 13, wherein said torso-worn belt comprises two electrodes to respectively contact skins at right side and at left side of a heart of the user, and said torso circuit further comprises an electrocardiac signal amplifier.

15. The method for obtaining a pulse transit time data according to claim 14, wherein said limb-worn module comprises a limb belt and a limb circuit which comprises a light source, a photo sensor, and an opto-electronic convertor.

16. The method for obtaining a pulse transit time data according to claim 15, wherein generating the pulse transit time data further comprises:
using the torso-worn module to wirelessly transmit the R-peak time to said limb-worn module;
using the limb-worn module to calculate the pulse transit time data according to the R-peak time and the pulse peak time,
wherein the method for obtaining a pulse transit time data further comprises using the limb-worn module to wirelessly transmit the pulse peak time to an information/telecommunication technological equipment.

17. The method for obtaining a pulse transit time data according to claim 15, wherein generating the pulse transit time data further comprises:
using the limb-worn module to wirelessly transmit the pulse peak time of the plethysmograph signal to said torso-worn module;
using the torso-worn module to calculate the pulse transit time according to the R-peak time and the pulse peak time,
wherein the method for obtaining a pulse transit time further comprises using the torso-worn module to wirelessly transmit the pulse peak time to an information/telecommunication technological equipment.

18. The method for obtaining a pulse transit time according to claim 16, further comprising said information/telecommunication technological equipment wirelessly transmitting the pulse transit time data to a remote monitoring center for storing and analyzing so as to direct the user or a care-giver of the user, wherein said information/telecommunication equipment is a cellular phone.

19. The method for obtaining a pulse transit time according to claim 16, further comprising said information/telecommunication technological equipment sending a sensory effect to guide the user to regulate respiration rhythm, wherein said information/telecommunication technological equipment is a cellular phone.

20. The method for obtaining a pulse transit time according to claim 13, further comprising:
using a blood pressure meter to calibrate the wearable physiological measurement device after providing the wearable physiological measurement device and before using the torso-worn module to acquired said R-peak time and before using the limb-worn module to acquired said pulse peak time.

21. The method for obtaining a pulse transit time data according to claim 13, further comprising:
turning on a warning message generator while a measured water content goes beyond a pre-determined range.

22. The wearable physiological measuring device according to claim 7, wherein said information/telecommunication technological equipment is configured to wirelessly transmit the pulse transit time data to a remote monitoring center for storing and analyzing so as to direct the user or a care-giver of the user, wherein said information/telecommunication technological equipment is a cellular phone.

23. The method for obtaining a pulse transit time according to claim 17, further comprising said information/telecommunication technological equipment wirelessly transmitting the pulse transit time data to a remote monitoring center for storing and analyzing so as to direct the user or a care-giver of the user, wherein said information/telecommunication equipment is a cellular phone.

24. The method for obtaining a pulse transit time according to claim 17, further comprising said information/telecommunication technological equipment sending a sensory effect to guide the user to regulate respiration rhythm, wherein said information/telecommunication technological equipment is a cellular phone.
